(19) United States
(12) Patent Application Publication
(10) Pub. No.: US 2010/0297264 A1
(43) Pub. Date: Nov. 25, 2010

(54) BOWEL PURGATIVE AND USES THEREOF

(75) Inventor: David Kastenberg, Haddonfield, NJ (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, Philadelphia, PA (US)

(21) Appl. No.: 12/682,897

(22) PCT Filed: Oct. 16, 2008
(86) PCT No.: PCT/US08/80116

§ 371 Date: Aug. 2, 2010

Related U.S. Application Data
(60) Provisional application No. 60/980,543, filed on Oct. 17, 2007.

(51) Int. Cl. A61K 31/695; A61K 33/14; A61K 33/04; A61K 33/00; A61P 1/00

(52) U.S. Cl. 424/680; 514/63; 424/709; 424/717

(57) ABSTRACT

The present invention is directed to a dry composition for admixture with water for oral administration to a mammal and methods of using the composition for cleansing the bowel of a mammal in need thereof.

This application claims benefit of U.S. Provisional Patent Application Ser. No. 60/980,543, filed Oct. 17, 2007, which is hereby incorporated by reference in its entirety.

### FIELD OF THE INVENTION

The present invention relates to a dry composition for admixture with water for use as a bowel purgative and methods of using the same.

### BACKGROUND OF THE INVENTION

Bowel cleansers, also called purgatives, cathartics, and lavages, are formulated for rapid emptying (cleansing) of the bowel. They are commonly used as “bowel preps” for emptying the bowel prior to surgery, childbirth, or diagnostic procedures, and usually comprise an osmotic or stimulant laxative administered by either the oral or anal route or both. While purgatives formulated for patient use as enemas are often prescribed before examinations, they are awkward to handle and are frequently not properly administered, or ineffective for cleansing the small bowel or large intestine beyond the area closest to the anus, so orally-administered preparations are generally preferred. However, the commonly used orally-administered compositions for rapid bowel cleansing also have disadvantages including large volumes to be ingested and unpleasant tastes which discourage patient compliance.

In attempts to avoid the problems associated with the high-volume type preparations, smaller-volume aqueous preparations consisting of phosphate salts have been marketed. The phosphate salt solution produces an osmotic effect, causing large amounts of water to be drawn into the bowel, thereby promoting bowel evacuation. Although the lower volume marginally favors these sodium phosphate preparations, adverse side effects such as nausea, vomiting (principally a result of unpalatable taste), abdominal bloating, pain and dizziness were of similar frequency compared to polyethylene glycol-electrolyte lavage. In addition, the use of sodium phosphate preparations is contraindicated for many medical conditions such as kidney disease or heart failure.

The most commonly prescribed oral bowel preps today for bowel examination comprise sodium phosphate compositions in varying proportions of mono- and dibasic species, and polyethylene glycol (PEG) in combination with electrolytes. For example, see U.S. Pat. No. 7,169,381, which is hereby incorporated by reference in its entirety.

Prior to 2001, examination of the small intestine was limited due to endoscopic inaccessibility. Wireless capsule endoscopy (WCE) is a new technology in which a vitamin-size capsule containing a camera is ingested and traverses the small bowel while taking two photos per second. WCE effectively and safely visualizes the small intestine, and in many respects is superior to traditional imaging with barium. This has been documented for small bowel diagnoses including bleeding (acute and chronic), tumors, and Crohn's disease. Until recently, Given Diagnostic Imaging System (Given) was the only company with an FDA approved wireless capsule (Pillcam®) capable of evaluating the small bowel. Another company, Olympus, has recently introduced a new wireless capsule. Over 500,000 PillCams have been ingested to date, and the market for this technology has grown 50% yearly. Technological advances are occurring rapidly in this field, and a wireless capsule for the colon is now undergoing testing.

Currently, WCE has only diagnostic utility, but efforts are under way to incorporate therapeutic functions as well. Furthermore, WCE is being considered for colorectal cancer screening, and for the diagnosis and treatment of colonic disorders, and such an indication would greatly increase the use of this technology.

As with any form of endoscopy, adequate cleansing of the mucosa is necessary for visualization. This is best exemplified in the colon purgative literature where inadequate cleansing prohibits adequate diagnosis of colonic neoplasia.

Cleansing is even more important for WCE, as there is no opportunity for flushing or suctioning as exists with colonoscopy. In the case of WCE, important clinical decisions are dependent on adequate visualization and interpretation of the findings. A number of recommendations may follow this procedure, including interventional endoscopy, surgery, medical therapy, further diagnostic testing, or observation. The current standard of care for preparing the small intestine for WCE, supported by the manufacturer, is a liquid diet after lunch until 10 pm on the day prior to the study, and fasting thereafter. Data from a few published studies, and several small studies published in abstract, suggests that this protocol is often inadequate.

The present invention is directed to overcoming these and other deficiencies in the art.

### SUMMARY OF THE INVENTION

One aspect of the present invention is directed to a dry composition for admixture with water. The dry composition comprises, per liter of aqueous solution to be made, the following components: 20 to 500 g polyethylene glycol, 0 to 20 g ascorbic acid, one or more salts of ascorbic acid, or a mixture of ascorbic acid and one or more salts of ascorbic acid, and 5 to 5000 mg simethicone.

Another aspect of the present invention is directed to a method of cleansing the intestine of a mammal. The method comprising administering orally to the mammal a cleansing fluid preparation comprising, per liter, the following components: 20 to 500 g polyethylene glycol, 0 to 20 g ascorbic acid, one or more salts of ascorbic acid, or a mixture of ascorbic acid and one or more salts of ascorbic acid, and 5 to 5000 mg simethicone.

### DETAILED DESCRIPTION OF THE INVENTION

One aspect of the present invention is directed to a dry composition for admixture with water. The dry composition comprises, per liter of aqueous solution to be made, the following components: 20 to 500 g polyethylene glycol, 0 to 20 g ascorbic acid, one or more salts of ascorbic acid, or a mixture of ascorbic acid and one or more salts of ascorbic acid, and 5 to 5000 mg simethicone.

In one embodiment of the dry composition, the simethicone component may be in the range of 20 to 1500 mg. In another embodiment, the simethicone component may be in the range of 80 to 1000 mg. The dry composition may also contain excipients such as flavoring, sweetener, or mixtures thereof. Also, the dry composition may further include electrolytes selected from the group consisting of sodium chloride, sodium sulfate, potassium chloride, sodium hydrogen carbonate, and mixtures thereof.

In certain embodiments, the dry composition comprises, per liter of aqueous solution to be made, the following components: 20 to 500 g polyethylene glycol and 5 to 5000 mg simethicone.

In another embodiments, the dry composition comprises, per liter of aqueous solution to be made, the following components: 20 to 500 g polyethylene glycol, 5 to 5000 mg simethicone, and 0 to 20 g ascorbic acid, one or more salts of ascorbic acid, or a mixture of ascorbic acid and one or more salts of ascorbic acid.

In yet another embodiment, the dry composition comprises, per liter of aqueous solution to be made, the following components: 20 to 500 g polyethylene glycol, 5 to 5000 mg simethicone, 0 to 20 g ascorbic acid, one or more salts of ascorbic acid, or a mixture of ascorbic acid and one or more salts of ascorbic acid, and electrolytes, for example, sodium chloride, sodium sulfate, potassium chloride, sodium hydrogen carbonate, and mixtures thereof.

One skilled in the art will recognize that any of the above compositions can optionally contain excipients such as flavoring, sweetener, or mixtures thereof. One skilled in the art will also recognize that the above compositions may be packaged within one or more containers such as pouches.

Another aspect of the present invention is directed to a method of cleansing the intestine of a mammal. This method involves administering orally to the mammal a cleansing fluid preparation comprising, per liter, the following components: 20 to 500 g polyethylene glycol, 0 to 20 g ascorbic acid, one or more salts of ascorbic acid, or a mixture of ascorbic acid and one or more salts of ascorbic acid, and 5 to 5000 mg simethicone. The cleansing preparation used in the method is substantially the same as the water admixture of the dry composition described above.

In one embodiment of the method, the volume of the total dose administered may be from 0.1 to 12 liters. In another embodiment the volume of the total dose administered may be from 0.5 to 8 liters. In still another embodiment the volume of the total dose administered may be from 1 to 4 liters. In one embodiment the total dose is consumed within a period of up to 24 hours prior to the start of the procedure to up to 24 hours after the start of the procedure.

The method of the present invention is preferably used to cleanse the intestine of the subject prior to, or during, a diagnostic, therapeutic, radiologic, or surgical procedure. Such procedures include, but are not limited to, endoscopy, including wireless capsule endoscopy; enteroscopy; wireless capsule colonoscopy; colonoscopy; radiologic evaluation; medical imaging; relief of constipation; and evacuation or removal of debris from the small bowel or colon lumen.

Those skilled in the art will recognize that the cleansing fluid preparation may be administered between 24 hours before the start to 24 hours after the start of the procedure and may be administered in one or more partial doses. In one embodiment the total dose is consumed within a period of 24 hours prior to the start of the procedure.

One skilled in the art will appreciate that the dry composition may be packaged in a single container or plurality of containers or pouches. A first container may contain polyethylene glycol, electrolytes such as sodium sulfate and sodium chloride, and excipients such as flavoring or sweeteners. A second container may contain ascorbic acid, one or more salts of ascorbic acid, or a mixture of ascorbic acid and one or more salts of ascorbic acid. The simethicone may be included in either of the aforementioned containers or may be contained in a separate container.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

### Exemplary Formulation 1

An exemplary formulation per one liter of water includes:

| | | |
|---|---|---|
| PEG | 100 | grams, molecular weight 3350, NF |
| Sodium sulfate | 7.5 | grams, USP |
| Sodium chloride | 2.691 | grams, USP |
| Potassium chloride | 1.015 | grams USP |
| Ascorbic acid | 4.7 | grams, USP |
| Sodium ascorbate | 5.9 | grams, USP |
| Simethicone | 5-5000 | mg |
| Excipients: | | |
| Aspartame, NF | | |
| Acesulfame potassium, NF | | |
| Lemon flavoring | | |

### Example 2

### Exemplary Formulation 2

Another formulation per one liter of water includes:

| | | |
|---|---|---|
| PEG | 105 | grams, molecular weight 3350, NF |
| Sodium chloride | 2.875 | grams, USP |
| Potassium chloride | 0.37 | grams, USP |
| Sodium bicarbonate | 1.43 | grams, USP |
| Simethicone | 5-5000 | mg |
| Excipients: | | |
| Flavoring | | |

### Example 3

### Exemplary Formulation 3

Another formulation per one liter of water includes:

| | | |
|---|---|---|
| PEG | 59.5 | grams, molecular weight 3350, NF |
| Sodium sulfate | 5.68 | grams, USP |
| Sodium chloride | 1.46 | grams, USP |
| Potassium chloride | 0.745 | grams, USP |
| Sodium bicarbonate | 1.68 | grams, USP |
| Simethicone | 5-5000 | mg |
| Excipients: | | |
| Flavoring | | |

### Example 4

### Exemplary Formulation 4

Another formulation per one liter of water or sugar-electrolyte solution includes:

| | |
|---|---|
| PEG | 225 grams, molecular weight 3350, NF |
| Simethicone | 5-5000 mg |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A dry composition for admixture with water wherein the dry composition comprises, per liter of aqueous solution to be made, the following components:
a) 20 to 500 g polyethylene glycol
b) 0 to 20 g ascorbic acid, one or more salts of ascorbic acid, or a mixture of ascorbic acid and one or more salts of ascorbic acid
c) 5 to 5000 mg simethicone.

2. The dry composition of claim 1 further comprising excipients selected from the group consisting of flavoring, sweetener, or mixtures thereof.

3. The dry composition of claim 1, wherein the simethicone component is in the range of 20 to 1500 mg.

4. The dry composition of claim 1, wherein the simethicone component is in the range of 80 to 1000 mg.

5. The dry composition of claim 1 further comprising electrolytes selected from the group consisting of sodium chloride, sodium sulfate, potassium chloride, sodium hydrogen carbonate, and mixtures thereof.

6. A method of cleansing the intestine of a mammal, comprising administering orally to the mammal a cleansing fluid preparation comprising, per liter, the following components:
a) 20 to 500 g polyethylene glycol
b) 0 to 20 g ascorbic acid, one or more salts of ascorbic acid, or a mixture of ascorbic acid and one or more salts of ascorbic acid
c) 5 to 5000 mg simethicone.

7. The method of claim 6, wherein the volume of a total dose of cleansing fluid preparation administered is from 0.1 liters to 12 liters.

8. The method of claim 6, wherein the volume of a total dose of cleansing fluid preparation administered is from 0.5 liters to 8 liters.

9. The method of claim 6, wherein the volume of a total dose of cleansing fluid preparation administered is from 1 liters to 4 liters.

10. The method of claim 6, wherein the intestine is cleansed prior to, or during, a diagnostic, therapeutic, radiologic, or surgical procedure.

11. The method of claim 10, wherein the procedure comprises wireless capsule endoscopy, endoscopy, enteroscopy, wireless capsule colonoscopy, colonoscopy, radiologic evaluation, medical imaging, relief of constipation, evacuation or removal of debris from the small bowel or colon lumen.

12. The method of claim 11, wherein the procedure is wireless capsule endoscopy.

13. The method of claim 6, wherein the cleansing fluid preparation is administered between 24 hours before to 24 hours after the start of the procedure.

14. The method of claim 6, wherein the cleansing fluid preparation total dose is administered in one or more partial doses.

15. The method of claim 14, wherein the cleansing fluid preparation total dose is administered in from one to six partial doses.

16. The composition of claim 1 for use in a method for cleansing the intestine of a mammal.

17. (canceled)
